(19) United States
(12) Patent Application Publication
(10) Pub. No.: US 2006/0210628 A1
(43) Pub. Date: Sep. 21, 2006

(54) Use of epinine for enhancing physical performance

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave, Detroit, MI, 48226, US

(21) Appl. No.: 11/385,073

(22) Filed: Mar. 21, 2006

Related U.S. Application Data
(60) Provisional application No. 60/663,608, filed on Mar. 21, 2005.

(51) Int. Cl. A61K 31/522; A61K 31/137; A61K 9/64; A61K 9/20

(52) U.S. Cl. 424/464; 514/649; 514/263.31; 514/60; 424/456

(57) ABSTRACT

A method of decreasing fat and adipose tissue, and increasing alertness in humans comprises administering epinine in a daily dosage between about 50 and 300 mg. Epinine may be administered once per day or in multiple doses, either alone or in combination with other compounds and mixtures.

### RELATED APPLICATION

This application claims priority benefit of U.S. provisional patent application No. 60/663,608 filed on Mar. 21, 2005.

### FIELD OF THE INVENTION

This invention relates to use of epinine as a supplement for enhancing physical performance.

### BACKGROUND OF THE INVENTION

Obesity is a growing concern, particularly in the United States. Excessive body weight can lead to a variety of well known health problems, especially hypertension, heart disease, atherosclerosis, diabetes and strokes. Weight loss, specifically fat loss, is critical in maintaining a healthy body weight. Unfortunately, the sedate modern lifestyle leaves people consuming more calories than they need.

In addition to diet control and exercise, there are several strategies for decreasing body weight through the use of pharmaceuticals and dietary supplements. Sympathomimetics, (stimulators of the sympathetic nerves) like ephedrine and amphetamines have been used extensively for this purpose. Although such compounds have been found effective, they are associated with side effects including increased heart rate and blood pressure.

Other strategies to decrease body weight include the use of compounds such as nicotine. Nicotine is known to stimulate acetylcholine receptors, and nicotine has been shown to have positive effects on weight loss. Unfortunately, the negative side effects of nicotine are well known and include addiction as well as carcinogenicity. It would be desirable to provide a new method for reducing weight, especially fat and adipose tissue, increasing endurance and increasing alertness.

### SUMMARY OF THE INVENTION

In accordance with a first aspect, a method of decreasing fat and adipose tissue and increasing endurance in humans comprises administering epinine in a daily dosage between about 50 and 300 mg.

From the foregoing disclosure and the following more detailed description of various preferred embodiments it will be apparent to those skilled in the art that the present invention provides a significant advance in the methods for reducing fat and adipose tissue and increasing endurance. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the methods of use of epinine disclosed here. The following detailed discussion of various alternative and preferred features and embodiments will illustrate the general principles of the invention with reference to improved methods of enhancing physical performance through the use of orally available dietary supplements. Other embodiments suitable for other applications will be apparent to those skilled in the art given the benefit of this disclosure.

The present invention provides a method of administering to mammals, especially humans, epinine or one of its derivatives, salts, extracts, tinctures or decoctations thereof, for maintaining or increasing endurance, stimulating lean muscle gain and/or decreasing fat mass.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance. Here, for example, the parent compound is epinine, or 4-(2-methylaminoethyl)benzene-1,2-diol. A derivative may be made by modification of the parent compound or by synthesis from other starting materials that are not similar to the parent.

Epinine, sometimes also referred to as N-methyl dopamine, is a naturally occurring compound which is a full agonist at dopamine (DA)-receptors and alpha- and beta-adrenoceptors. Epinine can be derived from several sources including, for example, the plant Portulaca oleracea. The chemical structure of epinine is reproduced below.

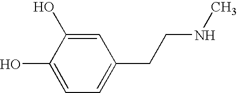

The method of using epinine to decrease body weight, especially reducing fat and adipose tissue, as well as increasing endurance and alertness is a significant improvement over the use of sympathomimetics, ephedrine, amphetamines and the like in that it can achieve the positive effects without the addictive side effects.

Administration of epinine to an individual provides numerous benefits, not just as an anti-obesity agent, but also as a thermogenic, a fatigue reliever and neural stimulant. Epinine can be administered one of the several ways: orally, transdermally, intranasally, by injection, sublingually, and transrectally as a suppository. Oral administration can comprise, for example, a liquid suspension, gel capsule or tablets. Tablets can be prepared by combining epinine with conventionally used binders and excipients, e.g. cellulose or cornstarch. Preferably a dosage would be about 50-300 mg per day, most preferably about 100-200 mg per day. Unit dosage can be about 50-100 mg or 25-50 mg of the compound, for example. Oral dosage forms could be administered in smaller divided doses multiple times per day (such as, two or three times per day) to help maintain more constant levels of epinine in the body.

Other compounds or mixtures may be added in combination with the epinine. For example caffeine or mixtures containing caffeine may be added, as caffeine help suppress appetite and aids in thermogenesis.

### EXAMPLE 1

Capsules

1 kg of the compound Epinine was mixed with cornstarch, and placed into 10000 hard-gelatin capsules each with a content of 100 mg of Epinine.

### EXAMPLE 2

Capsules

2 kg Epinine was mixed with 2 kg of caffeine, and placed into 10000 hard-gelatin capsules each with a content of 200 mg of Epinine and 200 mg of caffeine.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of decreasing fat and adipose tissue in humans comprising administering epinine in a daily dosage between about 50 mg and 300 mg.

2. The method of claim 1 wherein epinine is administered in one of the following ways: orally, transdermally, intranasally, by injection, sublingually, and transrectally.

3. The method of claim 1 wherein epinine is administered in a daily dosage between about 100 mg and 200 mg.

4. The method of claim 1 wherein epinine is administered in a dosage between about 50 mg and 100 mg three times per day.

5. The method of claim 1 wherein epinine is administered in a dosage between about 25 mg and 50 mg four times per day.

6. The method of claim 1 wherein the epinine is administered in the form of one of a gel capsule, a tablet and a liquid suspension.

7. The method of claim 1 wherein the epinine is administered in a tablet which also contains cornstarch.

8. The method claim 1 wherein the epinine is administered in combination with caffeine.
